(19) United States
(12) Patent Application Publication
(10) Pub. No.: US 2026/0226015 A1
(43) Pub. Date: Aug. 6, 2026

(54) COVALENT ORGANIC FRAMEWORK, AND PREPARATION METHOD AND USE THEREOF

(71) Applicant: Guizhou Education University, Guiyang (CN)

(72) Inventors: Qingqing WU, Guiyang (CN); Shuhao QIN, Guiyang (CN); Mingsen DENG, Guiyang (CN)

(73) Assignee: Guizhou Education University, Guiyang (CN)

(21) Appl. No.: 19/086,185

(22) Filed: Mar. 21, 2025

(30) Foreign Application Priority Data
Feb. 5, 2025 (CN) 202510128569.4

(51) Int. Cl. C07D 259/00

(52) U.S. Cl. CPC C07D 259/00

(57) ABSTRACT

Provided are a covalent organic framework (COF), and a preparation method and use thereof. The preparation method includes: mixing 2,4,6-triformylphloroglucinol, a polynary aromatic amine, and a polar organic solvent, and subjecting a resulting mixture to a Schiff base reaction and precipitation in sequence to obtain a crude product, where a ratio of a volume of the polar organic solvent to a mass of the 2,4,6-triformylphloroglucinol is in a range of 300 mL:1 g to 500 mL:1 g; and subjecting the crude product to washing and drying in sequence to obtain the COF.

### CROSS REFERENCE TO THE RELATED APPLICATIONS

This application is based upon and claims priority to Chinese Patent Application No. 202510128569.4, filed on Feb. 5, 2025, the entire contents of which are incorporated herein by reference.

### TECHNICAL FIELD

The present disclosure relates to the technical field of covalent organic frameworks, and in particular to a covalent organic framework, and a preparation method and use thereof.

### BACKGROUND

Covalent organic frameworks (COFs) have a wide range of applications in semiconductors, sensing, adsorption, membrane separation, energy storage, and heterogeneous catalysis due to their highly ordered pore sizes and designable pore structures. Currently, the COFs are mainly prepared by solvothermal method, ionothermal method, microwave method, and melt synthesis method. The solvothermal method is the most commonly used process for preparing COFs.

The solvothermal method involves adding a mixture of a precursor and a mixed solvent into a sealed reactor and conducting a reaction at certain temperature and pressure. However, the method has the following problems: First, the preparation of COFs by the solvothermal method requires anhydrous and oxygen-free sealing operations, which requires high equipment specifications. Second, the solvothermal reaction requires the precursor to form a solution, while the various precursors currently used have relatively poor solubility, requiring a large amount of organic solvents, which does not meet the requirements for green chemistry. Third, reaction conditions for the solvothermal method, such as temperature, pressure, solvent type and ratio, as well as acidity, are relatively complex, which may affect the formation of COFs. If not properly controlled, overreaction is likely to occur due to these factors, resulting in difficult separation and purification, reduced purity and crystallinity of the product, and leading to complex post-processing steps. Fourth, the solvothermal method requires a long reaction time, such as the preparation of COFs in the related prior art showing 2 days to 9 days of reaction at a temperature of 80° C. to 120° C. Fifth, many high-quality COFs can only be produced on a laboratory scale so far, making it difficult to achieve large-scale industrial production.

Therefore, there is an urgent need to provide a method for preparing COFs that is suitable for large-scale production, with simple equipment, environmental friendliness, and short reaction time.

### SUMMARY

An object of the present disclosure is to provide a COF, and a preparation method and use thereof. In the present disclosure, the preparation method does not require anhydrous and oxygen-free operations, has simple equipment, environmental friendliness, and short reaction time, and is suitable for large-scale production.

To achieve the above object, the present disclosure provides the following technical solutions:

The present disclosure provides a method for preparing a COF, including the following steps:
(1) mixing 2,4,6-triformylphloroglucinol, a polynary aromatic amine, and a polar organic solvent, and subjecting a resulting mixture to a Schiff base reaction and precipitation in sequence to obtain a crude product, where a ratio of a volume of the polar organic solvent to a mass of the 2,4,6-triformylphloroglucinol is in a range of 300 mL: 1 g to 500 mL: 1 g; and
(2) subjecting the crude product obtained in step (1) to washing and drying in sequence to obtain the COF.

In some embodiments, in step (1), a molar ratio of CHO in the 2,4,6-triformylphloroglucinol to NH₂ in the polynary aromatic amine is in a range of 1:1 to 1:1.2.

In some embodiments, in step (1), the polynary aromatic amine is at least one selected from the group consisting of a binary aromatic amine, a ternary aromatic amine, and a quaternary aromatic amine.

In some embodiments, the binary aromatic amine is selected from the group consisting of 4,4′-diaminooctafluorobiphenyl, 3,6-diaminocarbazole, 3,5-diaminopyridine, 2,5-diaminopyridine, 6,6′-diamino-2,2′-bipyridine, 2,4-diaminopyridine, 2,5-diaminobenzonitrile, 4,4′-diaminodiphenyl disulfide, 4,4′-diamino-2,2′-bipyridine, 2,4-diamino-1,3,5-triazine, 4,4′-diamino-[1,1′-biphenyl]-3-carbonitrile, benzidine, m-phenylenediamine, 1,4-xylylenediamine, m-xylylenediamine, 2,6-diaminopyridine, 4,4″-diamino-p-terphenyl, 3,5-diaminobenzotrifluoride, 2,3,5,6-tetramethyl-1,4-phenylenediamine, dianisidine, 2,5-dimethyl-1,4-phenylenediamine, 2,5-diaminotrifluorotoluene, 3,3′,5,5′-tetramethylbenzidine, 2,4,5,6-tetrafluoro-1,3-phenylenediamine, 4,4′-diamino-2,2′-dimethyl-1,l′-biphenyl, 4,4″-diaminoquaternary biphenyl, benzo[1,2-D: 4,5-D′]bis(thiazole)-2,6-diamine (CAS: 16162-28-0), 2,5-dimethyl-p-phenylenediamine, 3,3′-bis(allyloxy)-[1,1′-biphenyl]-4,4′-diamine (CAS: 2573217-06-6), 2,2′-difluoro-4,4′-diaminobiphenyl, 2,2′-bis(trifluoromethyl)benzidine, 2,2′-dichlorobenzidine, 2,2′-dibromo-4,4′-diaminobiphenyl, benzoguanamine, 6,7,9,10,17,18,20,21-octahydrodibenzo[b,k][1,4,7,10,13,16]hexaoxacyclooctadecane-2,13-diamine (CAS: 31406-52-7), methylguanamine, and 2-chloro-4,6-diamino-1,3,5-triazine;

the ternary aromatic amine is selected from the group consisting of tris(4-aminophenyl)amine, 1,3,5-tris(4-aminophenyl)benzene, 4,4′,4″-triaminotriphenylmethane, 1,3,5-tris(4-aminophenoxy)benzene, 2,4,6-tris(4-aminophenyl)-1,3,5-triazine, 1,3,5-tris[4′-amino (1,1-biphenyl-4-yl)]benzene (CAS: 1400987-00-9), 5′-(3-aminophenyl)-[1,1′: 3′,1″-terphenyl]-3,3″-diamine (CAS: 184650-02-0), and N4,N4-bis(4′-amino-[1,1′-biphenyl]-4-yl)-[1,l′-biphenyl]-4,4′-diamine; and
the quaternary aromatic amine is selected from the group consisting of 1,3,6,8-tetrakis-(p-aminophenyl)-pyrene, N,N,N′,N′-tetrakis(p-aminophenyl)-p-phenylenediamine, 4′,5′-bis(4-aminophenyl)-[1,1′: 2′,1″-terphenyl]-4,4″-diamine, and 5′,5″-bis(4-aminophenyl)-[1,1′: 3′,1″: 3″,1′″-quaterphenyl]-4,4′″-diamine.

In some embodiments, in step (1), the polar organic solvent is selected from the group consisting of 1,4-dioxane, N,N-dimethylformamide (DMF), ethylene glycol, n-butanol, isopropanol, ethanol, methanol, and tetrahydrofuran (THF).

In some embodiments, in step (1), the Schiff base reaction is conducted at a temperature of 20° C. to 150° C.

In some embodiments, in step (1), the Schiff base reaction is conducted for 5 h to 48 h.

In some embodiments, in step (2), a reagent for the washing is at least one selected from the group consisting of DMF, THE, and acetone.

The present disclosure further provides a COF prepared by the method as described above.

The present disclosure further provides use of the COF as described above in a solid electrolyte system.

The present disclosure provides a method for preparing a COF, including the following steps: mixing 2,4,6-triformylphloroglucinol, a polynary aromatic amine, and a polar organic solvent, and subjecting a resulting mixture to a Schiff base reaction and precipitation in sequence to obtain a crude product, where a ratio of a volume of the polar organic solvent to a mass of the 2,4,6-triformylphloroglucinol is in a range of 300 mL: 1 g to 500 mL: 1 g; and subjecting the crude product to washing and drying in sequence to obtain the COF. In the present disclosure, the mass of the 2,4,6-triformylphloroglucinol and the type range of the polar organic solvent are limited. The polar organic solvent does not need to completely dissolve the 2,4,6-triformylphloroglucinol. Instead, the dissolved portions of 2,4,6-triformylphloroglucinol and the polynary aromatic amine undergo a Schiff base reaction to form a Schiff base imine structure. Due to the presence of OH groups, the product is more readily converted into a stable keto structure, which is difficult to revert to the imine structure. As a result, the method provided by the present disclosure does not need to be operated under anhydrous and oxygen-free conditions. After the keto structure is generated, the 2,4,6-triformylphloroglucinol and the polynary aromatic amine continue to undergo a Schiff base reaction to supplement the generation of imine. At this time, the 2,4,6-triformylphloroglucinol dissolved in the polar organic solvent is consumed, and more 2,4,6-triformylphloroglucinol is dissolved in the solvent, thereby forming a dynamic process. Therefore, the method does not need to completely dissolve the 2,4,6-triformylphloroglucinol in the reaction system, which can significantly reduce the amount of organic solvent used, thus meeting the requirements for green chemistry. Moreover, reducing the amount of solvent and generating the stable keto structure serve as a driving force for the Schiff base reaction, and can shorten the reaction time and improve product purity. Moreover, the method does not require anhydrous and oxygen-free tube sealing to achieve desirable reaction results, has simple equipment requirements, and is suitable for large-scale production. The results of examples show that the method can produce gram-level crystalline materials within 5 h to 24 h, which is conducive to promoting the industrialization of COFs structure.

### BRIEF DESCRIPTION OF THE DRAWINGS

FIG. 1 shows the infrared spectra of the 2,4,6-triformylphloroglucinol, 4,4′-diaminooctafluorobiphenyl, and prepared COF-8F in Example 1 of the present disclosure;

FIG. 2 shows the scanning electron microscopy (SEM) image of the COF-8F prepared in Example 1 of the present disclosure;

FIG. 3 shows the infrared spectra of the 2,4,6-triformylphloroglucinol, 1,3,5-tris(4-aminophenyl)benzene, and prepared COF-TAPB in Example 2 of the present disclosure;

FIG. 4 shows the infrared spectra of the 2,4,6-triformylphloroglucinol, N,N,N′,N′-tetrakis(p-aminophenyl)-p-phenylenediamine, and prepared COF-PBABD in Example 3 of the present disclosure;

FIG. 5 shows the photographs of electrolyte sheets prepared in Use Example 1 and Comparative Use Example 1 of the present disclosure;

FIG. 6 shows the electrochemical window test results of the electrolyte prepared in Use Example 1 of the present disclosure;

FIG. 7 shows the electrochemical window test results of the electrolyte prepared in Comparative Use Example 1 of the present disclosure;

FIG. 8 shows the ionic conductivity test results of the electrolytes prepared in Use Example 1 and Comparative Use Example 1 of the present disclosure; and

FIG. 9 shows the cyclic stability test results of the electrolytes prepared in Use Example 1 and Comparative Use Example 1 of the present disclosure.

### DETAILED DESCRIPTION OF THE EMBODIMENTS

The present disclosure provides a method for preparing a COF, including the following steps:
(1) mixing 2,4,6-triformylphloroglucinol, a polynary aromatic amine, and a polar organic solvent, and subjecting a resulting mixture to a Schiff base reaction and precipitation in sequence to obtain a crude product, where a ratio of a volume of the polar organic solvent to a mass of the 2,4,6-triformylphloroglucinol is in a range of 300 mL: 1 g to 500 mL: 1 g; and
(2) subjecting the crude product obtained in step (1) to washing and drying in sequence to obtain the COF.

In the present disclosure, 2,4,6-triformylphloroglucinol, a polynary aromatic amine, and a polar organic solvent are mixed, and a resulting mixture is subjected to a Schiff base reaction and precipitation in sequence to obtain a crude product.

In some embodiments of the present disclosure, the polynary aromatic amine is at least one selected from the group consisting of a binary aromatic amine, a ternary aromatic amine, and a quaternary aromatic amine. The topological structure and crystallinity of the COF can be regulated by using the binary aromatic amine, the ternary aromatic amine, and the quaternary aromatic amine as reaction materials to react with the 2,4,6-triformylphloroglucinol, such that the COF shows excellent electrochemical properties when being used in an electrolyte system.

In some embodiments of the present disclosure, the binary aromatic amine is selected from the group consisting of 4,4′-diaminooctafluorobiphenyl, 3,6-diaminocarbazole, 3,5-diaminopyridine, 2,5-diaminopyridine, 6,6′-diamino-2,2′-bipyridine, 2,4-diaminopyridine, 2,5-diaminobenzonitrile, 4,4′-diaminodiphenyl disulfide, 4,4′-diamino-2,2′-bipyridine, 2,4-diamino-1,3,5-triazine, 4,4′-diamino-[1,l′-biphenyl]-3-carbonitrile, benzidine, m-phenylenediamine, 1,4-xylylenediamine, m-xylylenediamine, 2,6-diaminopyridine, 4,4″-diamino-p-terphenyl, 3,5-diaminotrifluorotoluene, 2,3,5,6-tetramethyl-1,4-phenylenediamine, dianisidine, 2,5-dimethyl-1,4-phenylenediamine, 2,5-diaminotrifluorotoluene, 3,3′,5,5′-tetramethylbenzidine, 2,4,5,6-tetrafluoro-1,3-phenylenediamine, 4,4′-diamino-2,2′-dimethyl-1,1′-biphenyl, 4,4″-diaminoquaternary biphenyl, benzo[1,2-D: 4,5-D′]bis(thiazole)-2,6-diamine, 2,5-dimethyl-p-phenylenediamine, 3,3′-bis(allyloxy)-[1,1′-biphenyl]-4,4′-diamine, 2,2′-difluoro-4,4′-diaminobiphenyl, 2,2′-bis(trifluoromethyl)benzidine, 2,2′-dichlorobenzidine, 2,2′-dibromo-4,4′-diaminobiphenyl, benzoguanamine, 6,7,9,10,17,18,20,21-octahydrodibenzo[b,k][1,4,7,10,13,16]hexaoxacyclooctadecane-2,13-diamine, methylguanamine, and 2-chloro-4,6-diamino-1,3,5-triazine.

In some embodiments of the present disclosure, the ternary aromatic amine is selected from the group consisting of tris(4-aminophenyl)amine, 1,3,5-tris(4-aminophenyl)benzene, 4,4′,4″-triaminotriphenylmethane, 1,3,5-tris(4-aminophenoxy)benzene, 2,4,6-tris(4-aminophenyl)-1,3,5-triazine, 1,3,5-tris[4′-amino (1,1-biphenyl-4-yl)]benzene, 5′-(3-aminophenyl)-[1,1′: 3′,1″-terphenyl]-3,3″-diamine, and N4,N4-bis(4′-amino-[1,1′-biphenyl]-4-yl)-[1,1′-biphenyl]-4,4′-diamine.

In some embodiments of the present disclosure, the quaternary aromatic amine is selected from the group consisting of 1,3,6,8-tetrakis-(p-aminophenyl)-pyrene, N,N,N′,N′-tetrakis(p-aminophenyl)-p-phenylenediamine, 4′,5′-bis(4-aminophenyl)-[1,1′: 2′,1″-terphenyl]-4,4″-diamine, and 5′,5″-bis(4-aminophenyl)-[1,1′: 3′,1″: 3″,1′″-quaterphenyl]-4,4″-diamine.

In some embodiments of the present disclosure, a molar ratio of CHO in the 2,4,6-triformylphloroglucinol to NH₂ in the polynary aromatic amine (hereinafter referred to asn(CHO):n (NH₂)) is in a range of 1:(1-1.2), more preferably 1:(1-1.1). Controlling the molar ratio ofn(CHO):n (NH₂) within the above range can regulate the crystallinity and stability of COFs, such that they show excellent electrochemical properties when being used in electrolyte systems.

In some embodiments of the present disclosure, the polar organic solvent is selected from the group consisting of 1,4-dioxane, N,N-dimethylformamide (DMF), ethylene glycol, n-butanol, isopropanol, ethanol, methanol, and tetrahydrofuran (THF), more preferably selected from the group consisting of DMF, ethylene glycol, n-butanol, isopropanol, and ethanol. The single polar organic solvent is used as a reaction solvent, which exhibits desirable solubility for the 2,4,6-triformylphloroglucinol and the polynary aromatic amine. Moreover, the single polar organic solvent can continue to dissolve the 2,4,6-triformylphloroglucinol after the 2,4,6-triformylphloroglucinol and the polynary aromatic amine are consumed by the reaction, thus forming a dynamic equilibrium process, reducing the amount of solvent used, prompting the reaction to quickly reach a dynamic equilibrium, and accelerating the reaction rate.

In the present disclosure, a ratio of a volume of the polar organic solvent to a mass of the 2,4,6-triformylphloroglucinol is in a range of 300 mL: 1 g to 500 mL: 1 g. As an embodiment, the ratio of the volume of the polar organic solvent to the mass of the 2,4,6-triformylphloroglucinol is 300 mL: 1 g, 350 mL: 1 g, 400 mL: 1 g, 450 mL: 1 g, or 500 mL: 1 g. The ratio of the volume of the polar organic solvent to the mass of the 2,4,6-triformylphloroglucinol is controlled within the above range, and the organic solvent can partially dissolve the 2,4,6-triformylphloroglucinol, such that the 2,4,6-triformylphloroglucinol dissolved in the solvent participates in the Schiff base reaction to form an imine, which further forms a thermodynamically more stable keto structure through tautomers. This process is an irreversible process. While the imine is converted into a more stable keto structure in the system, the equilibrium of the 2,4,6-triformylphloroglucinol and the polynary aromatic amine participating in the Schiff base reaction is transferred to a direction of generating imine. While one part of the 2,4,6-triformylphloroglucinol in the system is consumed, another part of the 2,4,6-triformylphloroglucinol is dissolved in the solvent, forming a continuous dynamic equilibrium process. This process reduces the amount of solvent used, thereby prompting the reaction to quickly reach a dynamic equilibrium, and then accelerating the reaction rate.

In some embodiments of the present disclosure, the 2,4,6-triformylphloroglucinol, the polynary aromatic amine, and the polar organic solvent are mixed by ultrasonication. The dissolution of the 2,4,6-triformylphloroglucinol in the polar organic solvent is accelerated by ultrasonication. In an embodiment, the ultrasonication is conducted for 5 min.

In some embodiments of the present disclosure, the Schiff base reaction is conducted at a temperature of 20° C. to 150° C.; as an embodiment, the Schiff base reaction is conducted at 50° C., 60° C., 70° C., 80° C., 90° C., 100° C., 110° C., 120° C., 130° C., 140° C., or 150° C. In some embodiments, the Schiff base reaction is conducted for 5 h to 48 h; as an embodiment, the Schiff base reaction is conducted for 5 h, 6 h, 8 h, 10 h, 12 h, 15 h, 18 h, 20 h, or 24 h. At the above temperature and time, it is more conducive to promoting the full reaction of the 2,4,6-triformylphloroglucinol and the polynary aromatic amine to obtain COFs.

In some embodiments of the present disclosure, the precipitation is conducted by cooling precipitation. In some embodiments, the cooling precipitation is conducted by cooling a system obtained after the Schiff base reaction to room temperature to form a precipitate. The COFs formed in the system after the Schiff base reaction are precipitated in the form of the precipitate by cooling.

In some embodiments of the present disclosure, under a condition that no precipitate is formed in the system after the Schiff base reaction is cooled to room temperature, water is added into the system after the Schiff base reaction which is cooled to room temperature. The COFs are precipitated by adding the water. There is no particular limitation on the amount of the water, which can be adjusted as needed to promote sufficient precipitation of the COFs. In an embodiment, a volume ratio of the water to the polar organic solvent is 10:1.

In some embodiments of the present disclosure, a system obtained by the precipitation is filtered to obtain a crude product. There is no particular limitation on a filtering method, and any conventional filtering method can be used to fully separate a solid obtained by the precipitation.

In the present disclosure, the crude product is washed and dried in sequence to obtain the COF.

In some embodiments of the present disclosure, a reagent for the washing is at least one selected from the group consisting of DMF, THE, and acetone, more preferably at least one selected from the group consisting of the THF and the acetone. Since the driving force for the Schiff base reaction is a small amount of polar organic solvent and the formation of a stable keto structure, the reaction product has a high purity and less impurities. Therefore, using the above reagent to wash the crude product can fully remove impurities without complicated post-processing operations.

In some embodiments of the present disclosure, the drying is conducted at a temperature of 30° C. to 100° C., more preferably 60° C. to 80° C. for 5 h to 24 h, more preferably 12 h to 24 h. In some embodiments, the drying is conducted by vacuum drying. In an embodiment, the vacuum drying is conducted under a vacuum degree of 27 MPa. The drying removes the residual washing reagent in the product COFs.

In the present disclosure, compared with the conventional solvent thermal synthesis in anhydrous and oxygen-free sealed tube, the method adopts a process that can be conducted in large amount and on a large scale in a flask. Moreover, the method avoids the harsh vacuum conditions in the conventional synthesis and can be conducted in any atmosphere, exhibiting simple process conditions, convenient operation, and benefits in large-scale production.

The present disclosure further provides a COF prepared by the method as described above.

In an embodiment of the present disclosure, the COF has the following structural formula:

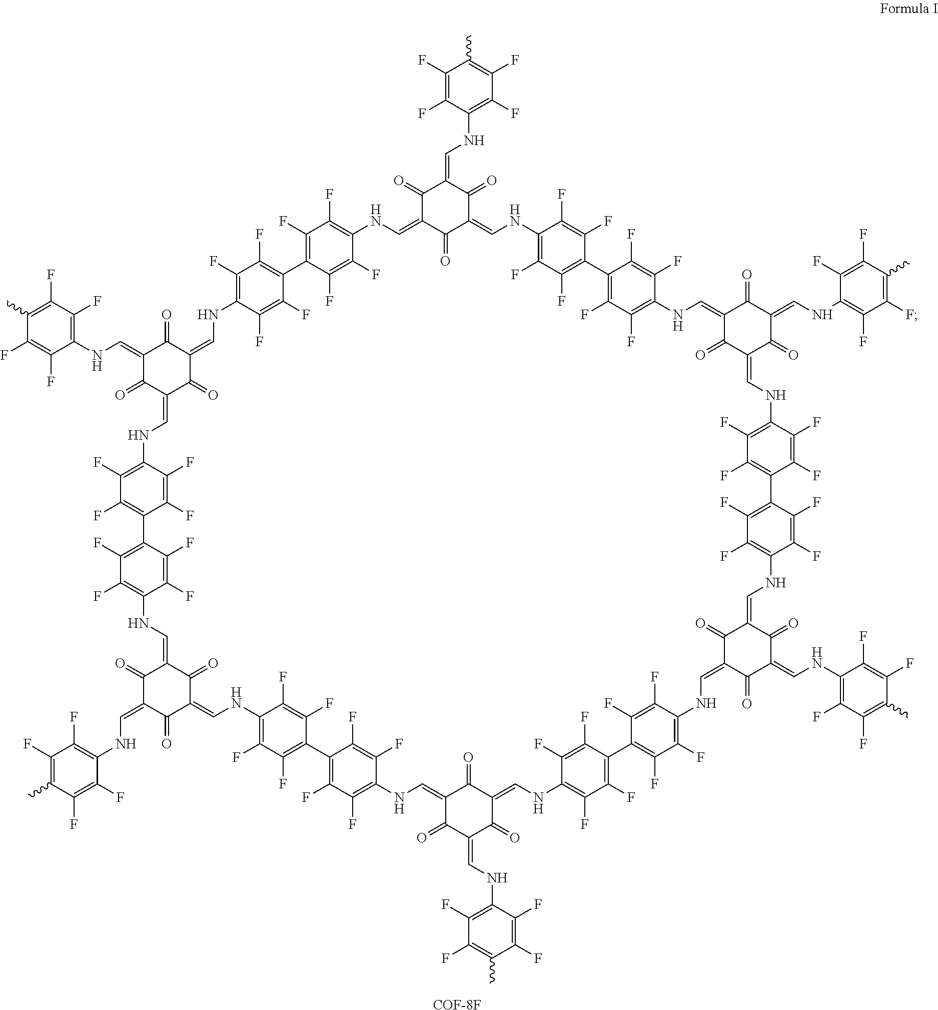

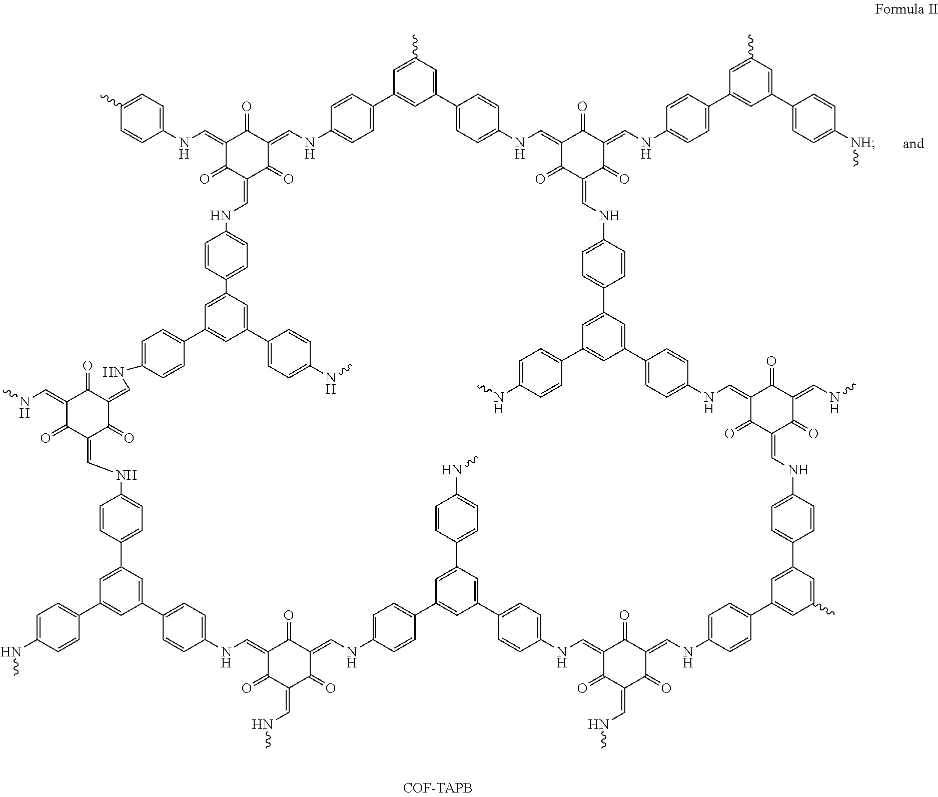

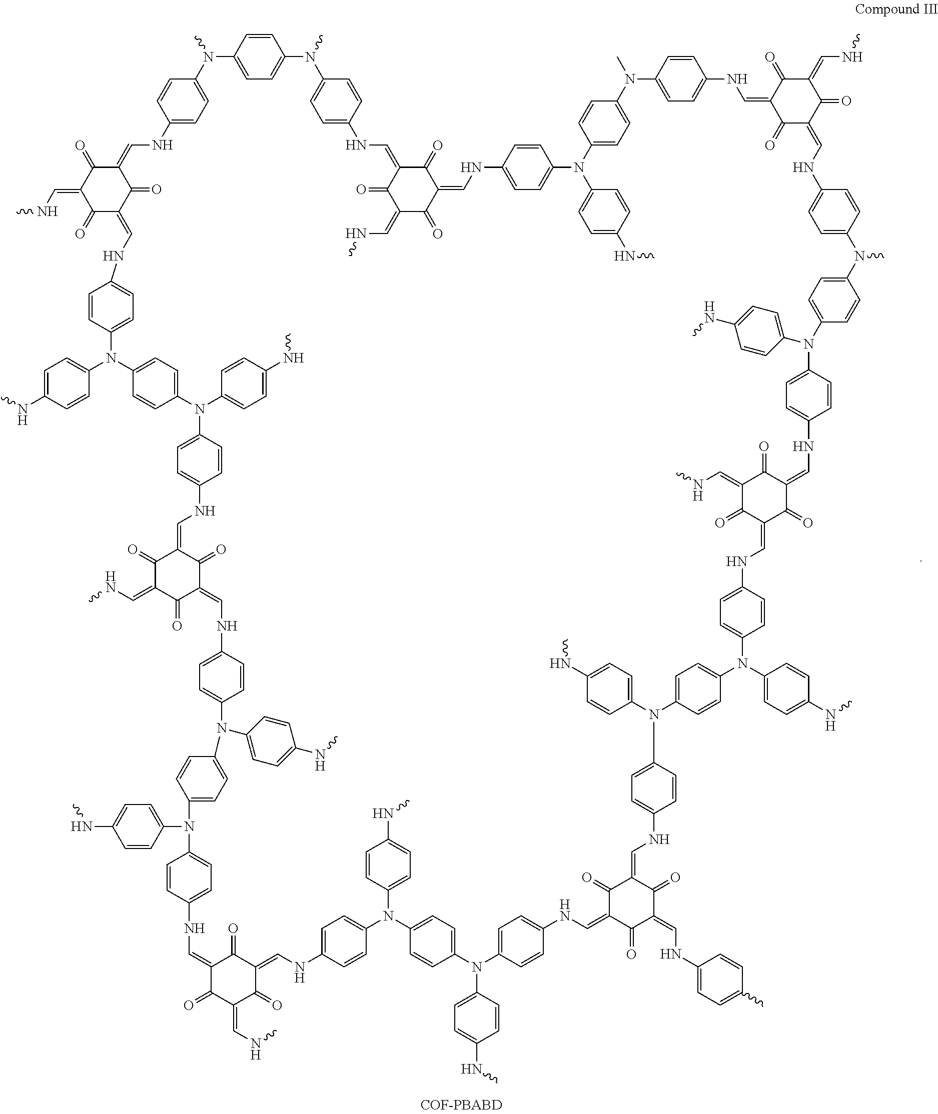

In some embodiments of the present disclosure, the COF has the above structural formula and exhibits a regular pore structure and stability.

The present disclosure further provides use of the COF as described above in a solid electrolyte system.

In the present disclosure, there is no specific limitation on a method for using the COF in an electrolyte system, and the COF can be added to the electrolyte system as an additive.

In the present disclosure, since the COF has a regular pore structure and stability, it can provide a fast conduction path for charge carriers such as lithium ions, which is beneficial to improving the ionic conductivity of the electrolyte and further improving the electrochemical performance of the electrolyte.

The technical solutions of the present disclosure will be clearly and completely described below with reference to the examples of the present disclosure. Apparently, the described examples are merely a part rather than all of the examples of the present disclosure. All other examples obtained by those skilled in the art based on the examples of the present disclosure without inventive effort shall fall within the scope of the present disclosure.

### Example 1

A method for preparing a COF was performed as follows:

(1) 2,4,6-triformylphloroglucinol (0.210 g, 1 mmol) and 4,4′-diaminooctafluorobiphenyl (0.541 g, 1.65 mmol) were added into a 500 mL round-bottom flask, and 105 mL of anhydrous ethanol was added thereto (where a ratio of a volume of the anhydrous ethanol to a mass of the 2,4,6-triformylphloroglucinol was 500 mL: 1 g), and a resulting mixture was ultrasonicated for 5 min, heated to 50° C. and then reacted for 8 h. A resulting reaction product was cooled and then filtered to obtain a crude product.

(2) The crude product obtained in step (1) was washed with THF 3 times (10 mL each time) and acetone 3 times (30 mL each time), and then dried in a vacuum drying oven (vacuum degree of 27 MPa) at 30° C. for 24 h to obtain a yellow COF, recorded as COF-8F.

A synthetic path was as follows:

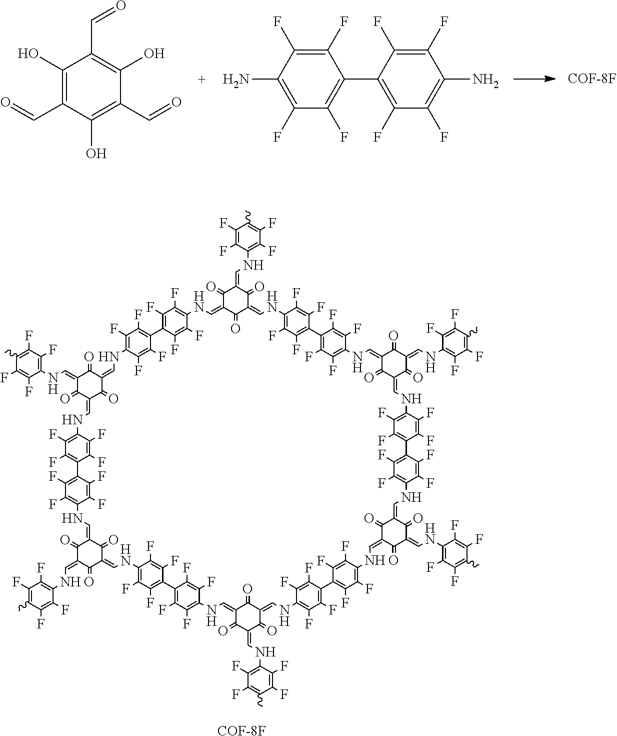

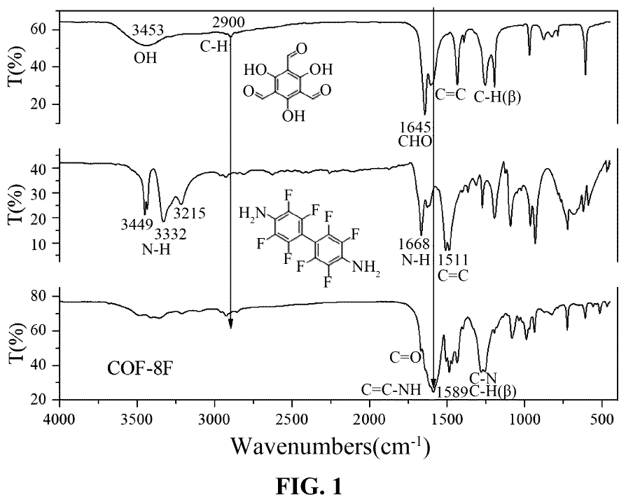

FIG. 1 shows the infrared spectra of the 2,4,6-triformylphloroglucinol, 4,4′-diaminooctafluorobiphenyl, and prepared COF-8F in this example. As shown in FIG. 1, the aldehyde C—H stretching vibration peak corresponding to 2,900 cm⁻¹ and the N—H stretching vibration peak corresponding to (3,200-3,500) cm⁻¹ disappear significantly, indicating that the aldehyde and amine undergo condensation. In addition, the disappearance of the broad hydroxyl stretching vibration peak at 3,453 cm⁻¹ and the appearance of a strong keto-enamine C═C double bond stretching vibration peak at 1,589 cm⁻¹ confirm the formation of keto-enamine bond in the COF-8F.

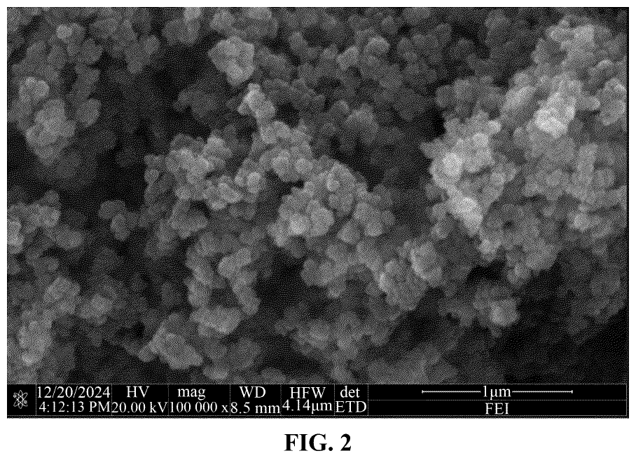

FIG. 2 shows the SEM image of the COF-8F prepared in this example. FIG. 2 proves that the COF-8F prepared in this example has a regular crystal structure.

### Example 2

A method for preparing a COF was performed as follows:

(1) 2,4,6-triformylphloroglucinol (0.210 g, 1 mmol) and 1,3,5-tris(4-aminophenyl)benzene (0.422 g, 1.2 mmol) were added into a 500 mL round-bottom flask, and 63 mL of DMF was added thereto (where a ratio of a volume of the DMF to a mass of the 2,4,6-triformylphloroglucinol was 300 mL: 1 g), and a resulting mixture was ultrasonicated for 5 min, heated to 150° C. and then reacted for 5 h. A resulting reaction solution was cooled and poured into 630 mL of water (10 times the amount of DMF), and a resulting solid was precipitated and filtered to obtain a crude product.

(2) The crude product obtained in step (1) was washed with DMF 3 times (5 mL each time) and acetone 3 times (30 mL each time), and then dried in a vacuum drying oven (vacuum degree of 27 MPa) at 100° C. for 24 h to obtain a yellow COF, recorded as COF-TAPB.

A synthetic path was as follows:

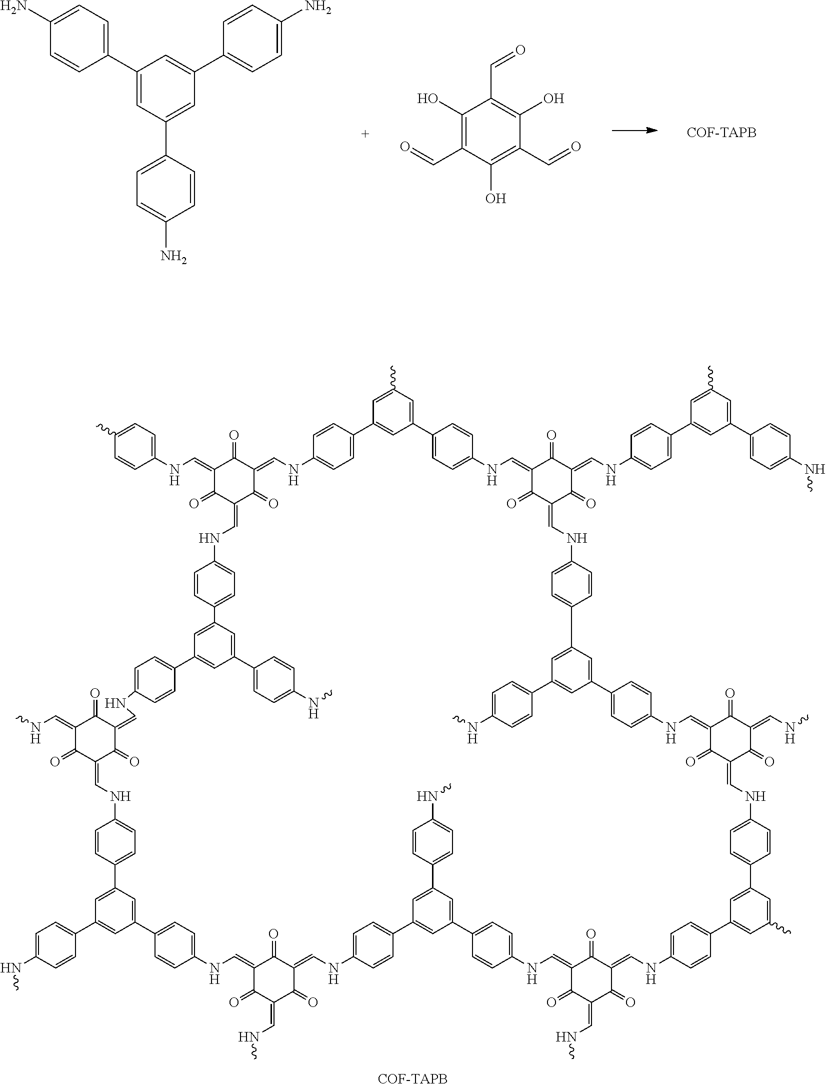

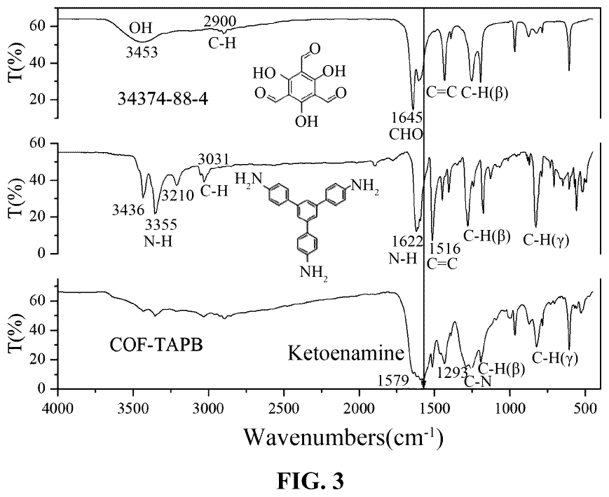

FIG. 3 shows the infrared spectra of the 2,4,6-triformylphloroglucinol, 1,3,5-tris(4-aminophenyl)benzene, and prepared COF-TAPB in this example. As shown in FIG. 3, the aldehyde C—H stretching vibration peak corresponding to 2,900 cm⁻¹ and the N—H stretching vibration peak corresponding to (3,200-3,500) cm⁻¹ disappear significantly, indicating that the aldehyde and amine undergo condensation. In addition, the disappearance of the broad hydroxyl stretching vibration peak at 3,453 cm⁻¹ and the appearance of a strong keto-enamine C═C double bond stretching vibration peak at 1,579 cm⁻¹ confirm the formation of keto-enamine bond in the COF-TAPB.

### Example 3

A method for preparing a COF was performed as follows:

(1) 2,4,6-triformylphloroglucinol (0.210 g, 1 mmol) and N,N,N′,N-tetrakis(p-aminophenyl)-p-phenylenediamine (0.354 g, 0.75 mmol) were added into a 500 mL round-bottom flask, and 73.5 mL of 1,4-dioxane was added thereto (where a ratio of a volume of the 1,4-dioxane to a mass of the 2,4,6-triformylphloroglucinol was 350 mL: 1 g), and a resulting mixture was ultrasonicated for 5 min, stirred at room temperature and then reacted for 24 h. A resulting reaction solution was poured into 735 mL of water (10 times the amount of dioxane), and a resulting solid was precipitated and filtered to obtain a crude product.

(2) The crude product obtained in step (1) was washed with THF 3 times (10 mL each time) and acetone 3 times (30 mL each time), and then dried in a vacuum drying oven (vacuum degree of 27 MPa) at 80° C. for 24 h to obtain a yellow COF, recorded as COF-PBABD.

A synthetic path was as follows:

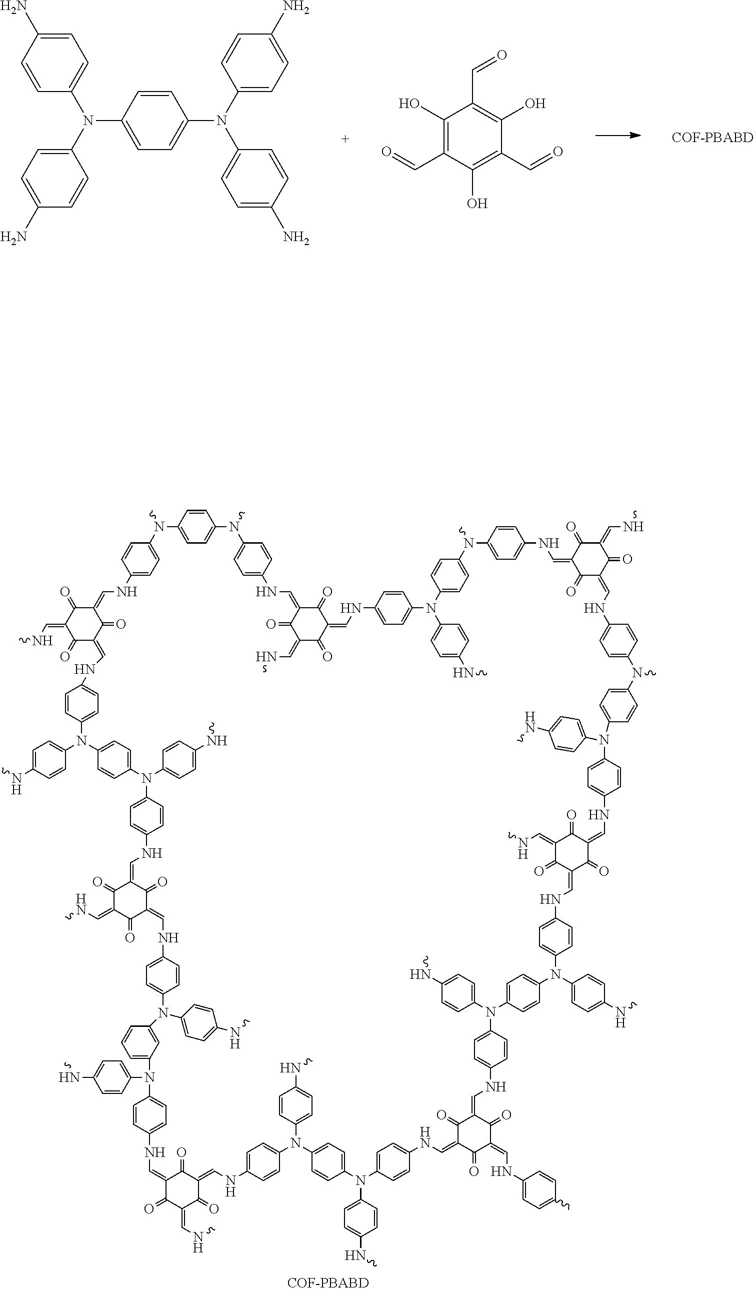

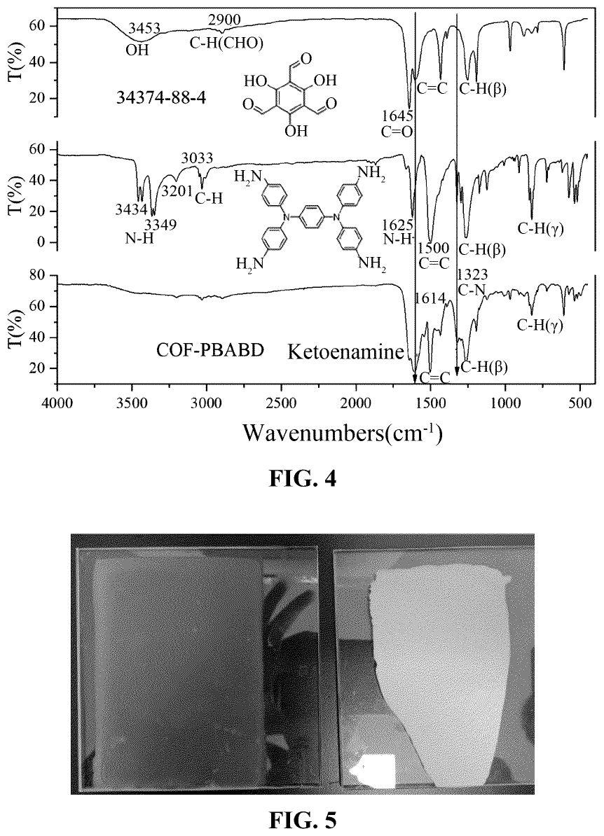

FIG. 4 shows the infrared spectra of the 2,4,6-triformylphloroglucinol, N,N,N′,N′-tetrakis(p-aminophenyl)-p-phenylenediamine, and prepared COF-PBABD in this example. As shown in FIG. 4, the aldehyde C—H stretching vibration peak corresponding to 2,900 cm⁻¹ and the N—H stretching vibration peak corresponding to (3,200-3,500) cm⁻¹ disappear significantly, indicating that the aldehyde and amine undergo condensation. In addition, the disappearance of the broad hydroxyl stretching vibration peak at 3,453 cm-1 and the appearance of a strong broad peak containing keto-enamine C═C double bond stretching vibration peak at 1,614 cm-1 confirm the formation of keto-enamine bond in the COF-PBABD.

### Use Example 1

A method for preparing an electrolyte material was performed as follows:

(1) 2 g of PVDF-HFP and 20 mL of DMF were added into a 100 mL round-bottom flask, and a resulting mixture was heated to 60° C. and stirred for 0.5 h. 2 g of lithium trifluoromethanesulfonyl imide was added thereto, and a resulting mixture was heated and stirred for another 0.5 h to mix well. 0.1 g of the COF-8F prepared in Example 1 was added thereto, and a uniform slurry was obtained after ultrasonic dispersion.

(2) An electrolyte membrane was prepared on a glass plate by a coating process and then dried at 150° C. for 24 h.

(3) The electrolyte membrane was cut into electrolyte sheets, as shown in the right figure of FIG. 5.

The electrolyte material was assembled into a battery in the stacking manner of lithium sheet//solid electrolyte membrane//positive electrode sheet, and the performance of the battery was tested.

### Comparative Use Example 1

A method for preparing an electrolyte material was performed as follows:

(1) 2 g of PVDF-HFP and 20 mL of DMF were added into a 100 mL round-bottom flask, and a resulting mixture was heated to 60° C. and stirred for 0.5 h. 2 g of lithium trifluoromethanesulfonyl imide was added thereto, and a resulting mixture was heated and stirred for another 0.5 h to mix well.

(2) An electrolyte membrane was prepared on a glass plate by a coating process and then dried at 150° C. for 24 h.

(3) The electrolyte membrane was cut into electrolyte sheets, as shown in the left figure of FIG. 5.

The electrolyte material was assembled into a battery in the stacking manner of lithium sheet//solid electrolyte membrane//positive electrode sheet, and the performance of the battery was tested.

### Test Example 1

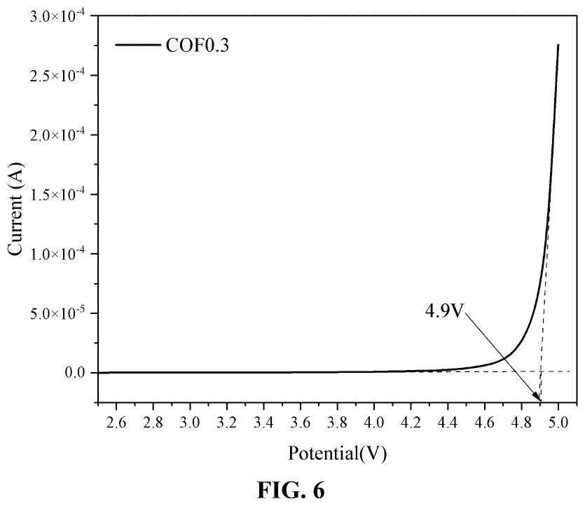

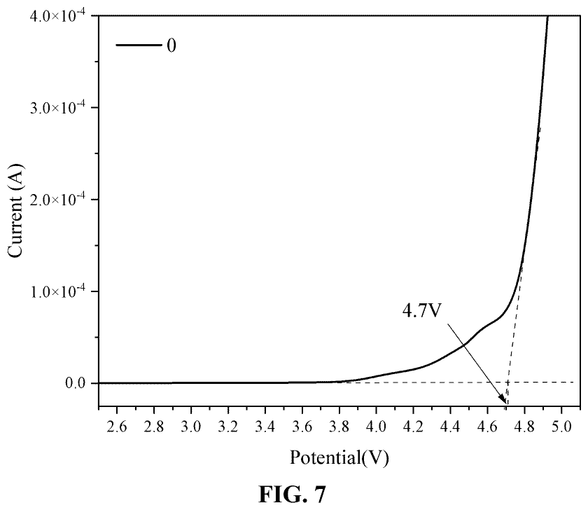

(1) The electrochemical window test results of the electrolyte prepared in Use Example 1 are shown in FIG. 6, while the test results of the electrolyte prepared in Comparative Use Example 1 are shown in FIG. 7. As shown in FIG. 6 and FIG. 7, the electrochemical window of the electrolyte is significantly improved with the addition of COF-8F.

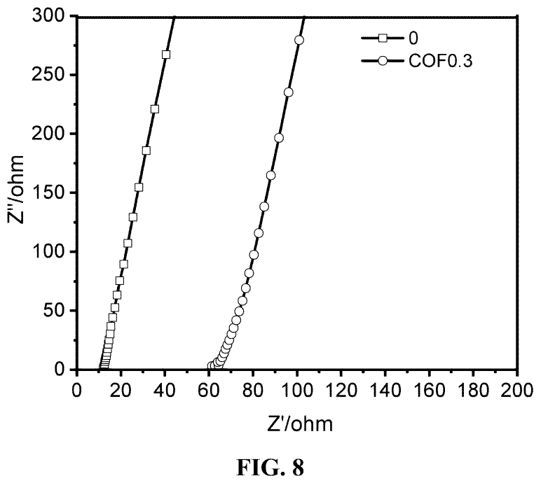

(2) The ionic conductivity test results of the electrolytes prepared in Use Example 1 and Comparative Use Example 1 are shown in FIG. 8. In FIG. 8, 0 represents the ionic conductivity test result of the electrolyte prepared in Comparative Use Example 1, and COF0.3 represents the ionic conductivity test result of the electrolyte prepared in Use Example 1. As shown in FIG. 8, the ionic conductivity of the electrolyte is significantly improved with the addition of COF-8F.

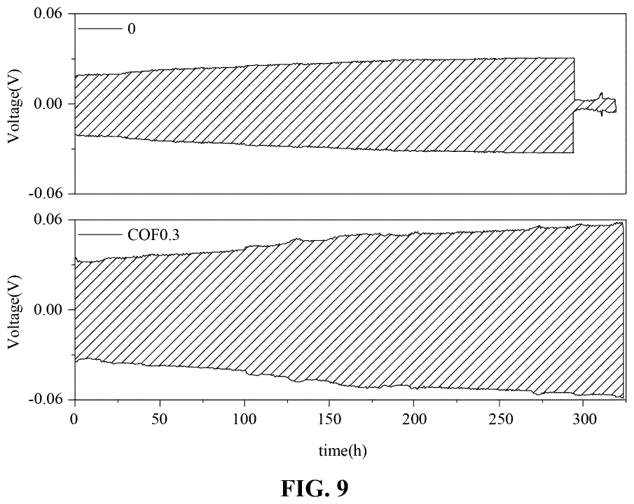

(3) The cyclic stability test results of the electrolytes prepared in Use Example 1 and Comparative Use Example 1 are shown in FIG. 9. In FIG. 9, 0 represents the cyclic stability test result of the electrolyte prepared in Comparative Use Example 1, and COF0.3 represents the cyclic stability test result of the electrolyte prepared in Use Example 1. As shown in FIG. 9, the cyclic stability of the electrolyte is significantly improved with the addition of COF-8F.

The results show that the addition of the COF in the present disclosure could significantly improve the electrochemical performance of the electrolyte. This is because the method in the present disclosure adopts a polar organic solvent to dissolve part of 2,4,6-triformylphloroglucinol, and the 2,4,6-triformylphloroglucinol reacts with polynary aromatic amine, and particles grow through polycondensation to produce increased particle size. The particles to be produced gradually increase in size, slowly precipitate, and then form a keto structure that is difficult to revert to an imine structure, such that the method in the present disclosure does not need to be operated under anhydrous conditions. By controlling the solubility and feed ratio of the COF structure in different solvents, the length of the COF chain when it precipitated from the solution can be controlled, and the molecular weight could be adjusted to have a regular pore structure. These pores provide a fast conduction path for charge carriers such as lithium ions, which is beneficial to improve the ionic conductivity of the electrolyte.

In addition, compared with the conventional solvent thermal synthesis in anhydrous and oxygen-free sealed tube, the method in the present disclosure adopts a process that could be conducted in large amount and on a large scale in a flask. Moreover, the method avoids the harsh vacuum conditions in the conventional synthesis and could be conducted in any atmosphere, exhibiting simple process conditions, convenient operation, and benefits in large-scale production.

The above descriptions are merely preferred embodiments of the present disclosure. It should be noted that those skilled in the art may further make several improvements and modifications without departing from the principle of the present disclosure, but such improvements and modifications should be deemed as falling within the scope of the present disclosure.

What is claimed is:

1. A method for preparing a covalent organic framework (COF), comprising the following steps:
(1) mixing 2,4,6-triformylphloroglucinol, a polynary aromatic amine, and a polar organic solvent, and subjecting a resulting mixture to a Schiff base reaction and precipitation in sequence to obtain a crude product, wherein a ratio of a volume of the polar organic solvent to a mass of the 2,4,6-triformylphloroglucinol is in a range of 300 mL: 1 g to 500 mL: 1 g; and
(2) subjecting the crude product obtained in the step (1) to washing and drying in sequence to obtain the COF.

2. The method of claim 1, wherein in the step (1), a molar ratio of CHO in the 2,4,6-triformylphloroglucinol to NH₂ in the polynary aromatic amine is in a range of 1:1 to 1:1.2.

3. The method of claim 1, wherein in the step (1), the polynary aromatic amine is at least one selected from the group consisting of a binary aromatic amine, a ternary aromatic amine, and a quaternary aromatic amine.

4. The method of claim 3, wherein the binary aromatic amine is selected from the group consisting of 4,4′-diaminooctafluorobiphenyl, 3,6-diaminocarbazole, 3,5-diaminopyridine, 2,5-diaminopyridine, 6,6′-diamino-2,2′-bipyridine, 2,4-diaminopyridine, 2,5-diaminobenzonitrile, 4,4′-diaminodiphenyl disulfide, 4,4′-diamino-2,2′-bipyridine, 2,4-diamino-1,3,5-triazine, 4,4′-diamino-[1,l′-biphenyl]-3-carbonitrile, benzidine, m-phenylenediamine, 1,4-xylylenediamine, m-xylylenediamine, 2,6-diaminopyridine, 4,4″-diamino-p-terphenyl, 3,5-diaminobenzotrifluoride, 2,3,5,6-tetramethyl-1,4-phenylenediamine, dianisidine, 2,5-dimethyl-1,4-phenylenediamine, 2,5-diaminotrifluorotoluene, 3,3′,5,5′-tetramethylbenzidine, 2,4,5,6-tetrafluoro-1,3-phenylenediamine, 4,4′-diamino-2,2′-dimethyl-1,l′-biphenyl, 4,4″-diaminoquaternary biphenyl, benzo[1,2-D: 4,5-D′]bis(thiazole)-2,6-diamine, 2,5-dimethyl-p-phenylenediamine, 3,3′-bis(allyloxy)-[1,1′-biphenyl]-4,4′-diamine, 2,2′-difluoro-4,4′-diaminobiphenyl, 2,2′-bis(trifluoromethyl)benzidine, 2,2′-dichlorobenzidine, 2,2′-dibromo-4,4′-diaminobiphenyl, benzoguanamine, 6,7,9,10,17,18,20,21-octahydrodibenzo[b,k][1,4,7,10,13,16]hexaoxacyclooctadecane-2,13-diamine, methylguanamine, and 2-chloro-4,6-diamino-1,3,5-triazine; the ternary aromatic amine is selected from the group consisting of tris(4-aminophenyl)amine, 1,3,5-tris(4-aminophenyl)benzene, 4,4′,4″-triaminotriphenylmethane, 1,3,5-tris(4-aminophenoxy)benzene, 2,4,6-tris(4-aminophenyl)-1,3,5-triazine, 1,3,5-tris[4′-amino (1,1-biphenyl-4-yl)]benzene, 5′-(3-aminophenyl)-[1,1′: 3′,1″-terphenyl]-3,3″-diamine, and N4,N4-bis(4′-amino-[1,l′-biphenyl]-4-yl)-[1,1′-biphenyl]-4,4′-diamine; and
the quaternary aromatic amine is selected from the group consisting of 1,3,6,8-tetrakis-(p-aminophenyl)-pyrene, N,N,N′,N′-tetrakis(p-aminophenyl)-p-phenylenediamine, 4′,5′-bis(4-aminophenyl)-[1,1′: 2′,1″-terphenyl]-4,4″-diamine, and 5′,5″-bis(4-aminophenyl)-[1,1′: 3′,1″: 3″,1′″-quaterphenyl]-4,4″-diamine.

5. The method of claim 1, wherein in the step (1), the polar organic solvent is selected from the group consisting of 1,4-dioxane, N,N-dimethylformamide (DMF), ethylene glycol, n-butanol, isopropanol, ethanol, methanol, and tetrahydrofuran (THF).

6. The method of claim 1, wherein in the step (1), the Schiff base reaction is conducted at a temperature of 20° C. to 150° C.

7. The method of claim 1, wherein in the step (1), the Schiff base reaction is conducted for 5 hours to 48 hours.

8. The method of claim 1, wherein in the step (2), a reagent for the washing is at least one selected from the group consisting of DMF, THE, and acetone.

9. A COF prepared by the method of claim 1.

10. The method of claim 6, wherein in the step (1), the Schiff base reaction is conducted for 5 hours to 48 hours.

11. The COF of claim 9, wherein in the step (1) of the method, a molar ratio of CHO in the 2,4,6-triformylphloroglucinol to NH₂ in the polynary aromatic amine is in a range of 1:1 to 1:1.2.

12. The COF of claim 9, wherein in the step (1) of the method, the polynary aromatic amine is at least one selected from the group consisting of a binary aromatic amine, a ternary aromatic amine, and a quaternary aromatic amine.

13. The COF of claim 12, wherein in the method, the binary aromatic amine is selected from the group consisting of 4,4′-diaminooctafluorobiphenyl, 3,6-diaminocarbazole, 3,5-diaminopyridine, 2,5-diaminopyridine, 6,6′-diamino-2,2′-bipyridine, 2,4-diaminopyridine, 2,5-diaminobenzonitrile, 4,4′-diaminodiphenyl disulfide, 4,4′-diamino-2,2′-bipyridine, 2,4-diamino-1,3,5-triazine, 4,4′-diamino-[1,1′-biphenyl]-3-carbonitrile, benzidine, m-phenylenediamine, 1,4-xylylenediamine, m-xylylenediamine, 2,6-diaminopyridine, 4,4″-diamino-p-terphenyl, 3,5-diaminobenzotrifluoride, 2,3,5,6-tetramethyl-1,4-phenylenediamine, dianisidine, 2,5-dimethyl-1,4-phenylenediamine, 2,5-diaminotrifluorotoluene, 3,3′,5,5′-tetramethylbenzidine, 2,4,5,6-tetrafluoro-1,3-phenylenediamine, 4,4′-diamino-2,2′-dimethyl-1, l′-biphenyl, 4,4″-diaminoquaternary biphenyl, benzo[1,2-D: 4,5-D′]bis(thiazole)-2,6-diamine, 2,5-dimethyl-p-phenylenediamine, 3,3′-bis(allyloxy)-[1,1′-biphenyl]-4,4′-diamine, 2,2′-difluoro-4,4′-diaminobiphenyl, 2,2′-bis(trifluoromethyl)benzidine, 2,2′-dichlorobenzidine, 2,2′-dibromo-4,4′-diaminobiphenyl, benzoguanamine, 6,7,9,10,17,18,20,21-octahydrodibenzo[b,k][1,4,7,10,13,16]hexaoxacyclooctadecane-2,13-diamine, methylguanamine, and 2-chloro-4,6-diamino-1,3,5-triazine;
the ternary aromatic amine is selected from the group consisting of tris(4-aminophenyl)amine, 1,3,5-tris(4-aminophenyl)benzene, 4,4′,4″-triaminotriphenylmethane, 1,3,5-tris(4-aminophenoxy)benzene, 2,4,6-tris(4-aminophenyl)-1,3,5-triazine, 1,3,5-tris[4′-amino (1,1-biphenyl-4-yl)]benzene, 5′-(3-aminophenyl)-[1,1′: 3′,1″-terphenyl]-3,3″-diamine, and N4,N4-bis(4′-amino-[1,1′-biphenyl]-4-yl)-[1,1′-biphenyl]-4,4′-diamine; and
the quaternary aromatic amine is selected from the group consisting of 1,3,6,8-tetrakis-(p-aminophenyl)-pyrene, N,N,N′,N′-tetrakis(p-aminophenyl)-p-phenylenediamine, 4′,5′-bis(4-aminophenyl)-[1,1′: 2′,1″-terphenyl]-4,4″-diamine, and 5′,5″-bis(4-aminophenyl)-[1,1′: 3′,1″: 3″,1′″-quaterphenyl]-4,4′″-diamine.

14. The COF of claim 9, wherein in the step (1) of the method, the polar organic solvent is selected from the group consisting of 1,4-dioxane, DMF, ethylene glycol, n-butanol, isopropanol, ethanol, methanol, and THF.

15. The COF of claim 9, wherein in the step (1) of the method, the Schiff base reaction is conducted at a temperature of 20° C. to 150° C.

16. The COF of claim 9, wherein in the step (1) of the method, the Schiff base reaction is conducted for 5 hours to 48 hours.

17. The COF of claim 9, wherein in the step (2) of the method, a reagent for the washing is at least one selected from the group consisting of DMF, THE, and acetone.
